(19) United States
(12) Patent Application Publication
(10) Pub. No.: US 2023/0360739 A1
(43) Pub. Date: Nov. 9, 2023

(54) METHOD AND APPARATUS OF PREDICTING SYNTHETIC PATH

(71) Applicant: SAMSUNG ELECTRONICS CO., LTD., Suwon-si (KR)

(72) Inventors: Jin Woo KIM, Suwon-si (KR); Youngchun Kwon, Suwon-si (KR); Dongseon Lee, Suwon-si (KR); Younsuk Choi, Suwon-si (KR); Joonhyuk Choi, Suwon-si (KR); Taesin Ha, Suwon-si (KR)

(73) Assignee: SAMSUNG ELECTRONICS CO., LTD., Suwon-si (KR)

(21) Appl. No.: 18/094,808

(22) Filed: Jan. 9, 2023

Related U.S. Application Data
(60) Provisional application No. 63/339,228, filed on May 6, 2022.

(30) Foreign Application Priority Data
Jun. 27, 2022 (KR) 10-2022-0078370

(51) Int. Cl. G16C 20/30; G16C 20/70; G06N 3/0455

(52) U.S. Cl. CPC G16C 20/30; G16C 20/70; G06N 3/0455

(57) ABSTRACT

A method of predicting a synthetic path may include: receiving a target molecule descriptor corresponding to a target material, predicting one or more synthetic path descriptor candidates representing multi-step synthetic paths corresponding to the target molecule descriptor using a neural network-based predictive model, the neural network-based predictive model being trained based on a synthetic path descriptor in a form of a character string obtained by converting multi-step synthetic path data; and outputting the one or more synthetic path descriptor candidates.

### CROSS-REFERENCE TO RELATED APPLICATION

This application claims priority from U.S. Provisional Application No. 63/339,228 filed on May 6, 2022, in the U.S. Patent and Trademark Office, and Korean Patent Application No. 10-2022-0078370, filed on Jun. 27, 2022, in the Korean Intellectual Property Office, the disclosures of which are incorporated herein by reference in their entireties.

### BACKGROUND

### 1. Field

Methods and apparatuses consistent with example embodiments relate to a predicting a synthetic path via artificial intelligence (AI)-based technologies.

### 2. Description of the Related Art

Designing synthetic paths may be important to achieve a high yield when synthesizing new materials. For example, the synthetic path may be manually determined by a synthesis expert, or may be determined in a manner of automatically predicting a synthetic path based on an algorithm using cumulative synthesis data.

However, in general, chemical reaction data may be expressed in single-step reaction units. In addition, even in a method of predicting a synthetic path through an algorithm, the synthetic path may be generally suggested in single-step units. For example, an algorithm may suggest candidate reactants N times for each synthesis step to retrieve a multi-step synthetic path. In this example, if the maximum number of retrieval steps is M, multi-step path combinations may be retrieved NM times, which may result in inefficiencies in processing load and/or processing time.

### SUMMARY

One or more example embodiments may address at least the above problems and/or disadvantages and other disadvantages not described above. Also, the example embodiments are not required to overcome the disadvantages described above, and an example embodiment may not overcome any of the problems described above.

According to an aspect of an example embodiment, there is provided a method of predicting a synthetic path, the method including receiving a target molecule descriptor corresponding to a target material, predicting one or more synthetic path descriptor candidates representing multi-step synthetic paths corresponding to the target molecule descriptor using a neural network-based predictive model, the neural network-based predictive model being trained based on a synthetic path descriptor obtained by converting multi-step synthetic path data, and outputting the one or more synthetic path descriptor candidates.

The synthetic path descriptor may include one or more tokens among a molecular structure descriptor representing molecular structure information of reactants, a notation character for distinguishing synthesis steps before and after synthesis of the multi-step synthetic paths, a delimiting parenthesis for defining a synthesis order of the reactants used for each synthesis step of the multi-step synthetic paths, a separator for distinguishing between the molecular structure descriptor and the delimiting parenthesis or distinguishing between a plurality of molecular structure descriptors, and a reaction descriptor corresponding to a reaction scheme used for each synthesis step of the multi-step synthetic paths.

The molecular structure descriptor may include a token representing at least one of types of atoms included in the reactants, bonding information including a bonding structure of the atoms, an aromatic compound corresponding to the molecular structure information, or an isomer corresponding to the molecular structure information.

The neural network-based predictive model may include an encoder and a decoder that are trained using the synthetic path descriptor in which a portion of tokens are masked.

The neural network-based predictive model may include a first encoder configured to extract embedding information from the target molecule descriptor, a second encoder that is trained to extract a first feature from the embedding information and a sequence including first tokens of a portion among tokens constituting the synthetic path descriptor, and a decoder that is trained to restore a character string corresponding to the synthetic path descriptor based on the first feature and information on second tokens other than the first tokens among the tokens constituting the synthetic path descriptor. The neural network-based predictive model may be trained by updating one or more weights of the neural network-based predictive model based on a difference between the synthetic path descriptor and the character string.

The second encoder may be configured to extract the first feature from a first sequence in which tokens of the portion among the tokens constituting the synthetic path descriptor are randomly masked, extract the first feature from a second sequence in which the tokens constituting the synthetic path descriptor are masked in units of delimiting parentheses for defining a synthesis order, or extract the first feature from a third sequence in which the tokens constituting the synthetic path descriptor are masked in units of molecular structure descriptors.

The neural network-based predictive model may include a first predictive model that receives a first target molecule descriptor corresponding to the target material and predicts the synthetic path descriptor corresponding to the target material and a second predictive model that receives the synthetic path descriptor and predicts a second target molecule descriptor corresponding to the target material.

The predicting of the one or more synthetic path descriptor candidates may include applying the first target molecule descriptor to the first predictive model, acquiring the synthetic path descriptor predicted by the first predictive model based on the first target molecule descriptor, determining whether a candidate material corresponding to the synthetic path descriptor is a starting material of which a chemical characteristic and a structure are known, predicting the second target molecule descriptor by applying the synthetic path descriptor to the second predictive model in response to a determination that the candidate material is the starting material, and predicting the one or more synthetic path descriptor candidates based on whether the first target molecule descriptor matches the second target molecule descriptor.

The applying of the first target molecule descriptor to the first predictive model may include classifying one or more characters representing an atom type of the target material and one or more characters representing a chemical bond of the target material in the first target molecule descriptor as tokens, and applying at least a portion of the tokens to the first predictive model.

The determining of whether the candidate material is the starting material may include determining whether the candidate material is the starting material using data registered in a material database.

The method may further include removing, in response to a determination that the candidate material is not the starting material, the synthetic path descriptor corresponding to the candidate material from the synthetic path descriptor candidates.

The method may further include providing a synthesis recipe corresponding to the target material based on the synthetic path descriptor candidates.

The method may further include receiving the multi-step synthetic path data corresponding to chemical materials and converting the multi-step synthetic path data into the synthetic path descriptor in a form of a character string.

According to an aspect of an example embodiment, there is provided a non-transitory computer-readable storage medium storing instructions that, when executed by at least one processor, cause the at least one processor to perform the method of predicting a synthetic path.

According to an aspect of an example embodiment, there is provided an apparatus for predicting a synthesis path, the apparatus including a user interface configured to receive a target molecule descriptor corresponding to a target material, at least one memory in which at least one program is stored, and at least one processor configured to executing the at least one program to: predict one or more synthetic path descriptor candidates representing multi-step synthetic paths corresponding to the target molecule descriptor using a neural network-based predictive model, the neural network-based predictive model being trained based on a synthetic path descriptor obtained by converting multi-step synthetic path data and provide a synthesis recipe corresponding to the target material based on the synthetic path descriptor candidates.

The synthetic path descriptor may include one or more tokens among a molecular structure descriptor representing molecular structure information of reactants, a notation character for distinguishing synthesis steps before and after synthesis of the multi-step synthetic paths, a delimiting parenthesis for defining a synthesis order of the reactants used for each synthesis step of the multi-step synthetic paths, a separator for distinguishing between the molecular structure descriptor and the delimiting parenthesis or distinguishing between a plurality of molecular structure descriptors, and a reaction descriptor corresponding to a reaction scheme used for each synthesis step of the multi-step synthetic paths.

The neural network-based predictive model may include an encoder and a decoder that are trained using the synthetic path descriptor in which a portion of tokens are masked.

The neural network-based predictive model may include a first encoder configured to extract embedding information from the target molecule descriptor, a second encoder that is trained to extract a first feature from the embedding information and a sequence including first tokens of a portion among tokens constituting the synthetic path descriptor, and a decoder that is trained to restore a character string corresponding to the synthetic path descriptor based on the first feature and information on second tokens other than the first tokens among the tokens constituting the synthetic path descriptor. The predictive model may be trained using a weight based on a difference between the synthetic path descriptor and the character string.

The neural network-based predictive model may include a first predictive model that receives a first target molecule descriptor corresponding to the target material and predicts the synthetic path descriptor corresponding to the target material and a second predictive model that receives the synthetic path descriptor and predicts a second target molecule descriptor corresponding to the target material.

The at least one processor may be configured to apply the first target molecule descriptor to the first predictive model, acquire the synthetic path descriptor predicted by the first predictive model based on the first target molecule descriptor, determine whether a candidate material corresponding to the synthetic path descriptor is a starting material of which a chemical characteristic and a structure are known, predict the second target molecule descriptor by applying the synthetic path descriptor to the second predictive model in response to a determination that the candidate material is the starting material, and predict the one or more synthetic path descriptor candidates based on whether the first target molecule descriptor matches the second target molecule descriptor.

Additional aspects of example embodiments will be set forth in part in the description which follows and, in part, will be apparent from the description, or may be learned by practice of the disclosure.

### BRIEF DESCRIPTION OF THE DRAWINGS

The above and/or other aspects will be more apparent by describing certain example embodiments with reference to the accompanying drawings, in which:

FIG. 1 is a block diagram illustrating an apparatus for predicting a synthetic path according to an example embodiment;

FIG. 2 is a diagram illustrating an input and an output of an apparatus for predicting a synthetic path according to an example embodiment;

FIG. 3 is a flowchart illustrating a method of predicting a synthetic path according to an example embodiment;

FIG. 4 is a diagram illustrating a multi-step synthetic path descriptor according to an example embodiment;

FIG. 5 is a diagram illustrating a multi-step synthetic path descriptor according to another example embodiment;

FIG. 6 is a diagram illustrating a process of training a predictive model in advance according to an example embodiment;

FIG. 7 is a diagram illustrating a method of training a predictive model according to an example embodiment;

FIG. 8 is a diagram illustrating a process of predicting a synthetic path using a trained predictive model according to an example embodiment;

FIG. 9 is a flowchart illustrating a method of predicting one or more synthetic path descriptor candidates according to an example embodiment;

FIG. 10 is a diagram illustrating a method of predicting one or more synthetic path descriptor candidates by a prediction apparatus according to an example embodiment; and

FIG. 11 is a diagram illustrating operations computed in a first predictive model and a second predictive model according to an example embodiment.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

Although terms of “first” or “second” are used to explain various components, the components are not limited to the terms. These terms should be used only to distinguish one component from another component. For example, a “first” component may be referred to as a “second” component, or similarly, and the “second” component may be referred to as the “first” component within the scope of the right according to the concept of the present disclosure.

It will be understood that when a component is referred to as being “connected to” another component, the component can be directly connected or coupled to the other component or intervening components may be present.

As used herein, the singular forms are intended to include the plural forms as well, unless the context clearly indicates otherwise. It should be further understood that the terms “comprises” and/or “comprising,” when used in this specification, specify the presence of stated features, integers, steps, operations, elements, components or a combination thereof, but do not preclude the presence or addition of one or more other features, integers, steps, operations, elements, components, and/or groups thereof.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

Unless otherwise defined herein, all terms used herein including technical or scientific terms have the same meanings as those generally understood by one of ordinary skill in the art. Terms defined in dictionaries generally used should be construed to have meanings matching with contextual meanings in the related art and are not to be construed as an ideal or excessively formal meaning unless otherwise defined herein.

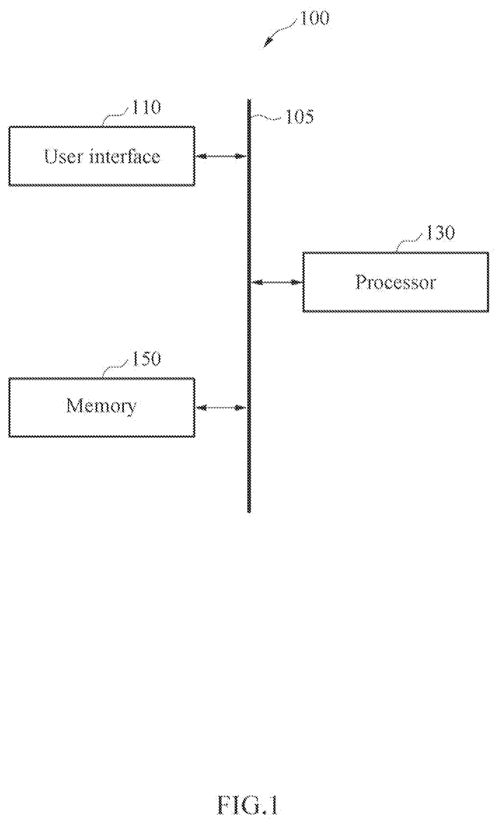

FIG. 1 is a block diagram illustrating an apparatus for predicting a synthetic path according to an example embodiment. Referring to FIG. 1, an apparatus 100 for predicting a synthetic path according to an example embodiment (hereinafter, referred to as a “prediction apparatus”) may include a user interface 110, a processor 130, and a memory 150. The user interface 110, the processor 130, and the memory 150 may be connected to one another through a communication bus 105.

The user interface 110 may receive a target molecule descriptor corresponding to a target material. The “target material” may correspond to a material to be generated through a synthetic path. The target material may also be referred to as a “target molecule.” Hereinafter, the target material and the target molecule may be understood as having the same meaning.

The user interface 110 may include, for example, a keypad, a dome switch, a touchpad, a jog wheel, a jog switch, and a graphic user interface, but is not limited thereto. For the touchpad, various methods may be used in addition to, for example, a contact-type capacitive method, a pressure-type resistive film method, an infrared sensing method, a surface ultrasonic conduction method, an integral tension measurement method, and a piezo effect method.

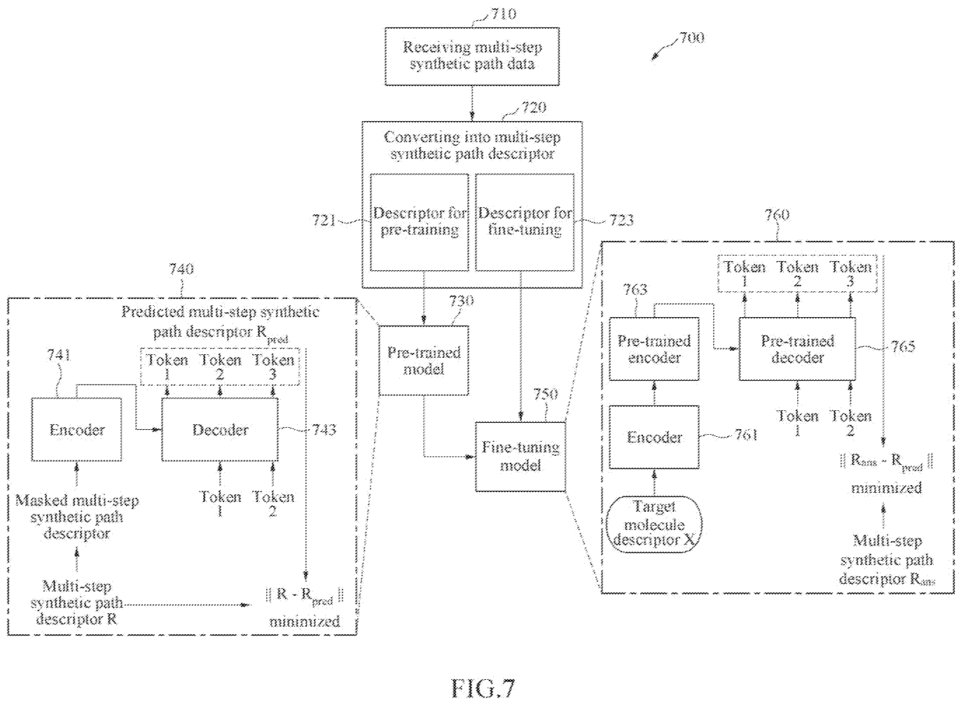

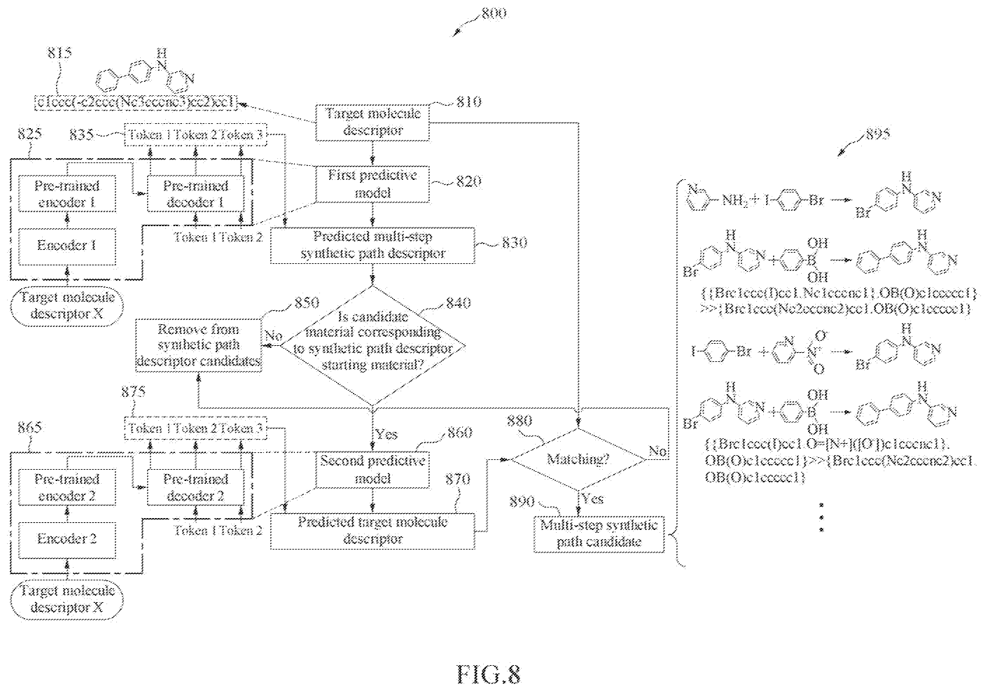

The processor 130 may run a neural network-based predictive model by executing at least one program stored in the memory 150. The processor 130 may predict one or more synthetic path descriptor candidates representing multi-step synthetic paths corresponding to the target molecule descriptor received through the user interface 110, using a previously trained neural network-based predictive model (e.g., a pre-trained model 740 of FIG. 7 and/or a first predictive model 825 and a second predictive model 865 of FIG. 8) stored in the memory 150. The neural network-based predictive model may be trained based on a synthetic path descriptor in a form of a character string obtained by converting multi-step synthetic path data.

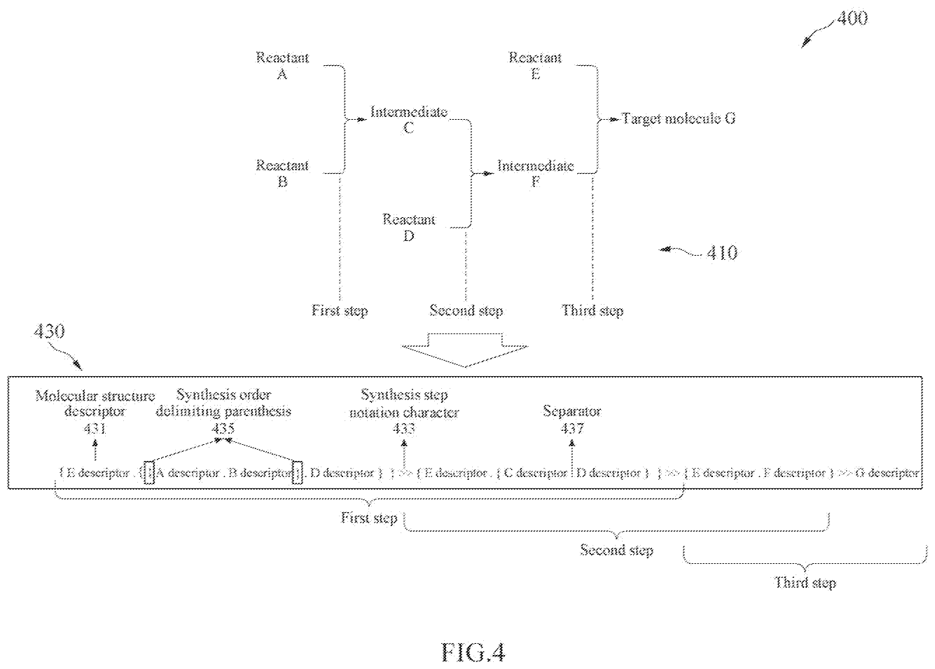

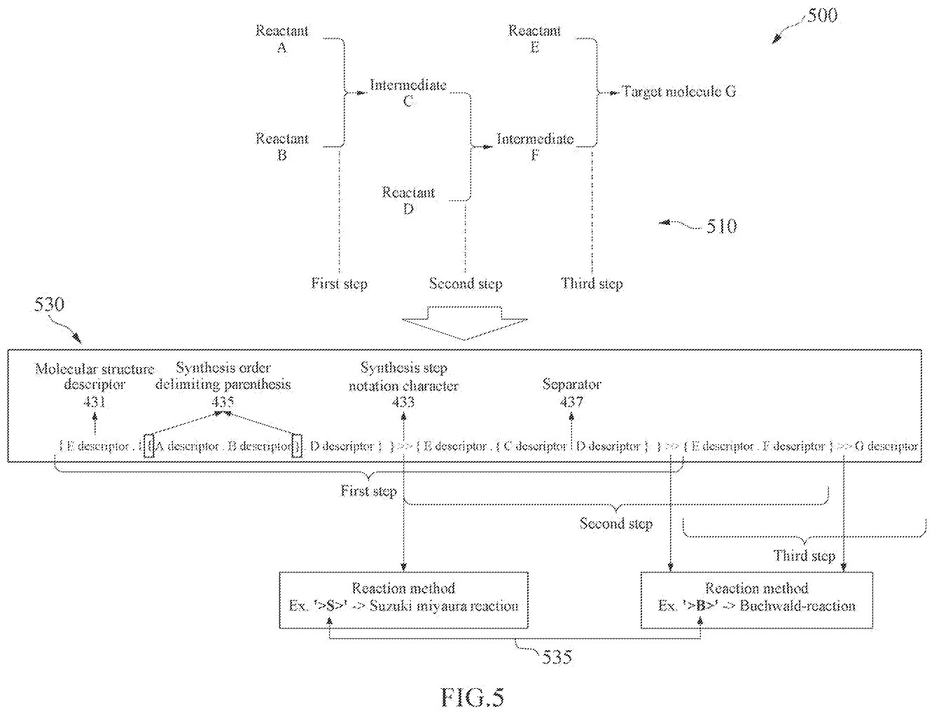

The synthetic path descriptor may include one or more tokens, for example, among a molecular structure descriptor representing molecular structure information of reactants, a notation character for distinguishing synthesis steps before and after synthesis of the multi-step synthetic paths, a delimiting parenthesis for defining a synthesis order of the reactants used for each synthesis step of the multi-step synthetic paths, a separator for distinguishing between the molecular structure descriptor and the delimiting parenthesis or distinguishing between a plurality of molecular structure descriptors, and a reaction descriptor corresponding to a reaction scheme used for each synthesis step of the multi-step synthetic paths. In addition, the molecular structure descriptor may include a token representing at least one of types of atoms included in the reactants, bonding information including a bonding structure of the atoms, an aromatic compound corresponding to the molecular structure information, or an isomer corresponding to the molecular structure information. The synthetic path descriptor and tokens constituting the synthetic path descriptor will be described in greater detail with reference to FIGS. 4 and 5.

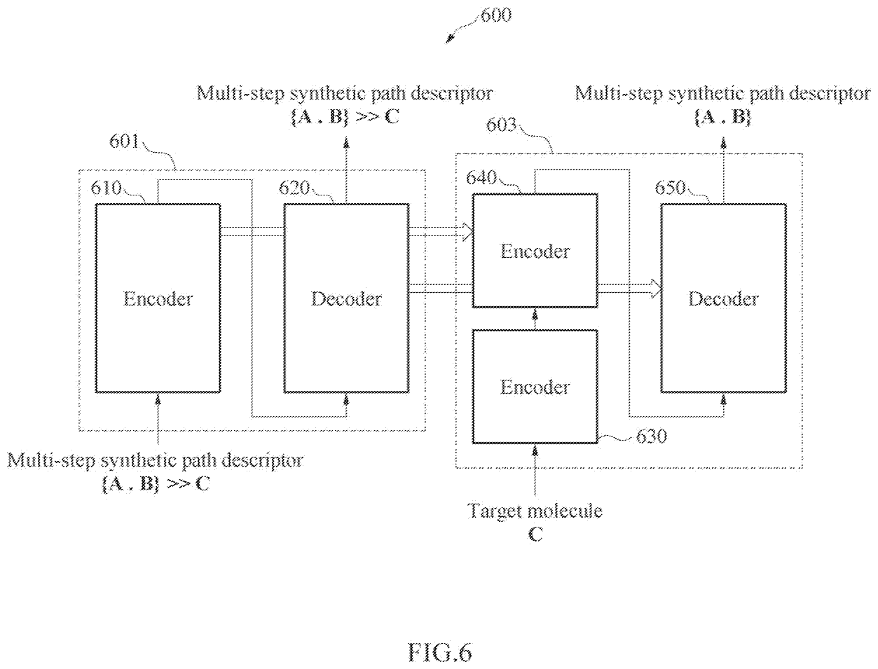

The predictive model may include, for example, an encoder and a decoder previously trained using the synthetic path descriptor in which a portion of tokens are masked. The predictive model may include, for example, a first encoder (e.g., an encoder 630 of FIG. 6 and/or an encoder 761 of FIG. 7) that extracts embedding information from the target molecule descriptor, a second encoder (e.g., an encoder 640 of FIG. 6 and/or a pre-trained encoder 763 of FIG. 7) previously trained to extract a first feature from the embedding information and a sequence including first tokens of a portion among tokens constituting the synthetic path descriptor, and a decoder (e.g., a decoder 650 of FIG. 6 and/or a pre-trained decoder 765 of FIG. 7) previously trained to restore a character string corresponding to the synthetic path descriptor based on the first feature extracted by the second encoder and information on second tokens other than the first tokens among the tokens constituting the synthetic path descriptor. The predictive model may be trained using a weight based on a difference between the synthetic path descriptor and the character string restored by the previously trained decoder. A method of training the predictive model according to an example embodiment will be described in greater detail with reference to FIGS. 6 and 7.

The predictive model may include a first predictive model (e.g., a first predictive model 825 of FIG. 8) that receives a first target molecule descriptor corresponding to the target material and predicts the synthetic path descriptor corresponding to the target material and a second predictive model (e.g., a second predictive model 865 of FIG. 8) that receives the synthetic path descriptor and predicts a second target molecule descriptor corresponding to the target material.

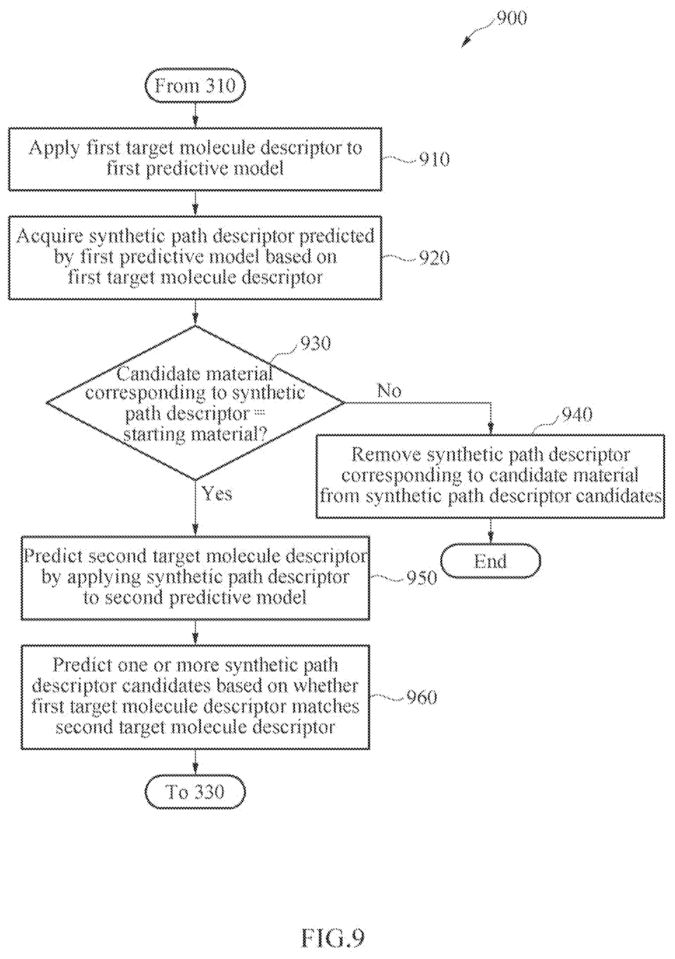

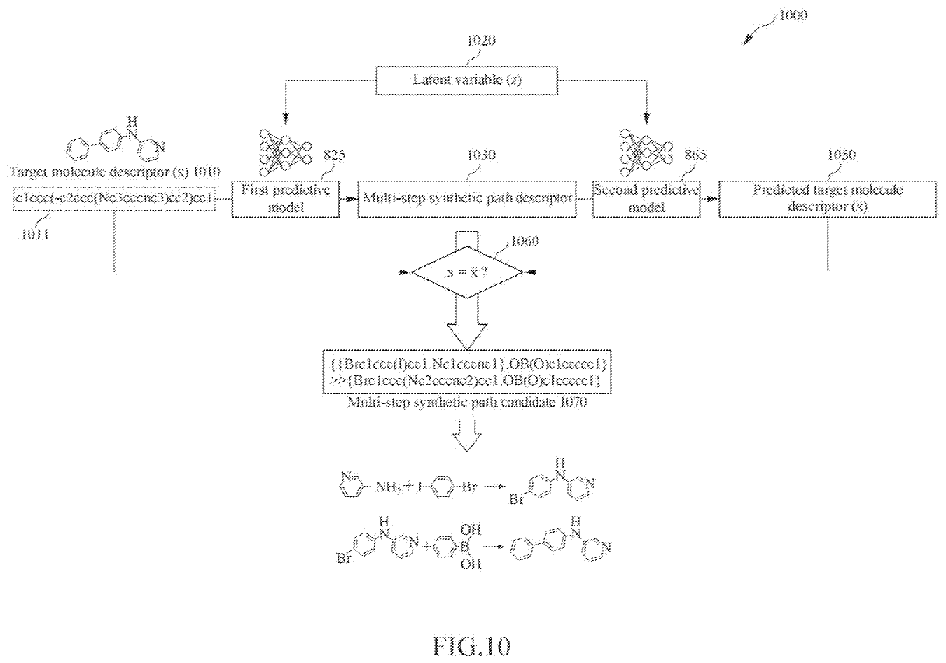

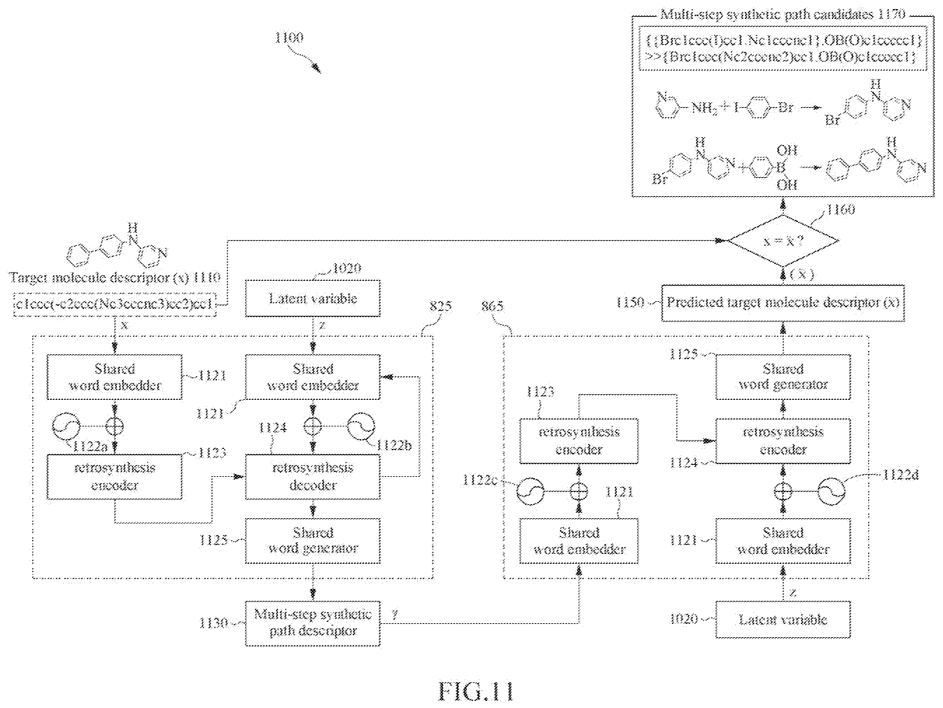

The processor 130 may apply the first target molecule descriptor to the first predictive model and acquire a synthetic path descriptor predicted by the first predictive model based on the first target molecule descriptor. For example, the processor 130 may classify one or more characters representing an atom type of the target material and one or more characters representing a chemical bond of the target material in the first target molecule descriptor as tokens. The processor 130 may apply at least a portion of the tokens to the first predictive model. The processor 130 may determine whether a candidate material corresponding to the synthetic path descriptor is a starting material of which a chemical characteristic and a structure are known. In response to a determination that the candidate material is not the starting material, the processor 130 may remove a synthetic path descriptor corresponding to the candidate material from the synthetic path descriptor candidates. In response to a determination that the candidate material is the starting material, the processor 130 may predict the second target molecule descriptor by applying the synthetic path descriptor to the second predictive model. The processor 130 may predict the one or more synthetic path descriptor candidates based on whether the first target molecule descriptor matches the second target molecule descriptor. A process of predicting synthetic path descriptor candidates by the prediction apparatus 100 according to an example embodiment will be described in detail with reference to FIGS. 8 and 9. In addition, configurations and operations of predictive models included in the prediction apparatus 100 will be described in detail with reference to FIGS. 10 and 11.

The processor 130 may provide a synthesis recipe corresponding to the target material based on the synthetic path descriptor candidates. Hereinafter, the “synthetic path descriptor candidate” may also be referred to as a “synthetic path candidate.”

The processor 130 may execute executable instructions included in the memory 150. When the instructions are executed in the processor 130, the processor 130 may load the neural network-based predictive model stored in the memory 150 and may input the target molecule descriptor corresponding to the target material to the predictive model. The processor 130 may execute a program and may control the prediction apparatus 100. Codes of the program executed by the processor 130 may be stored in the memory 150.

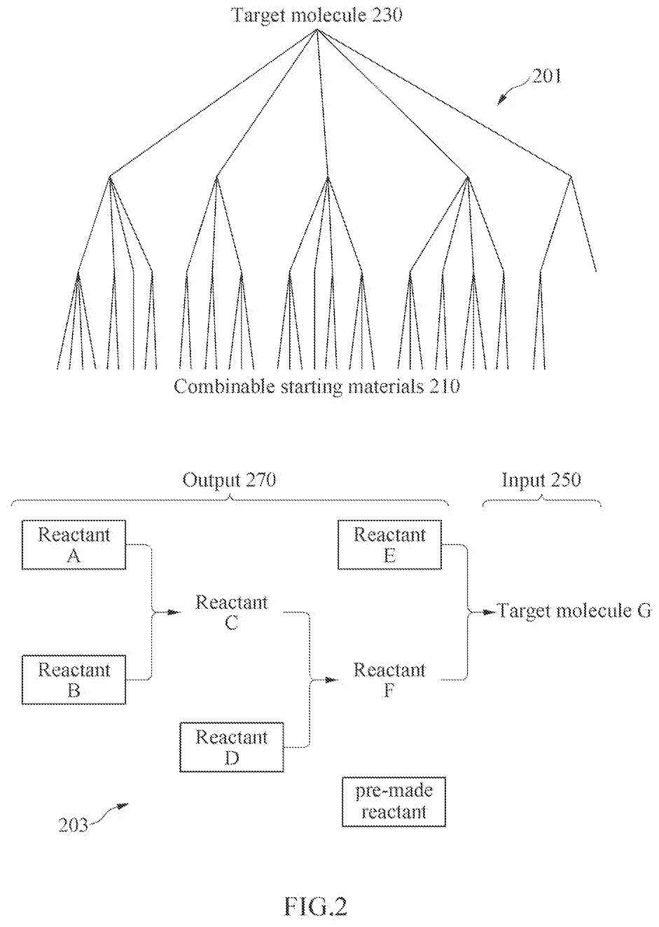

Further, the processor 130 may perform at least one method described with reference to FIGS. 2 through 11 in addition to FIG. 1 and a technique corresponding to the at least one method. The processor 130 may be a hardware-implemented prediction apparatus having a circuit that is physically structured to execute desired operations. For example, the desired operations may include code or instructions included in a program. The processor 130 may include, for example, a microprocessor, a central processing unit (CPU), a graphics processing unit (GPU), a processor core, a multi-core processor, a multiprocessor, an application-specific integrated circuit (ASIC), a field-programmable gate array (FPGA), a neural processing unit (NPU), and a machine learning accelerator. The processor 130 may refer to a single processor, a multi-core processor, or a plurality of processors.

The memory 150 may store at least one program. In addition, the memory 150 may store a variety of information generated in a processing operation of the processor 130. The memory 150 may store a neural network-based predictive model. Further, the memory 150 may store a variety of data and programs, and the like. The memory 150 may include a volatile memory or a nonvolatile memory. The memory 150 may include a massive storage medium such as a hard disk to store a variety of data.

FIG. 2 is a diagram illustrating an input and an output of an apparatus for predicting a synthetic path according to an example embodiment. FIG. 2 illustrates a part 201 that shows a process of generating a target material (e.g., a target molecule G) 230 by diversely combining multiple combinable starting materials 210 through a plurality of synthetic paths according to an example embodiment and a part 203 that shows synthetic paths between reactants (e.g., reactants A, B, C, D, E, and F) output as an output 270 through a retrosynthetic process in response to the target molecule G 230 being applied as an input 250 of a prediction apparatus according to an example embodiment.

In the part 201, the target molecule 230 may be generated by combining the multiple starting materials 210 through various synthetic paths. In contrast, as shown in the part 203, predicting various synthetic paths between reactants for generating the target molecule 230 (e.g., the target molecule G) inversely from the target molecule G to be generated may be referred to as a “retrosynthetic process.”

When the target molecule G is input, for example, a predictive model according to an example embodiment may predict various synthetic paths between reactants (e.g., reactants A, B, C, D, E, and F) for generating the target molecule G through the retrosynthetic process learned by a neural network-based predictive model. In particular, the reactants may include, for example, the combinable starting materials 210, a product produced through a chemical reaction between the starting materials, and/or precursor but are not limited thereto. Here, the target molecule may be a known material and may also be an unknown material.

The “reactant(s)” may correspond to a material participating in the chemical reaction, and the “product” may correspond to a material generated as a result of the chemical reaction. In other words, when one material changes into another material through a chemical reaction, the material(s) that react with each other are called reactants, and a material that is changed or newly formed through the chemical reaction may be called a product. The reactant may refer to a reaction material, and the product may refer to an output material.

The “starting material” may correspond to a component of which a chemical characteristic and a structure are known, and an unisolated intermediate product (e.g., an intermediate reactant) may not correspond to the starting material. The starting material may refer to, for example, a drug substance, intermediates and/or other drug substances used in the production of drug substances and becoming an important structural part of the drug substance. Here, the “important structural part” may be used to distinguish the starting material from reagents, solvents, and other raw materials. For example, common chemicals used to make salts, esters, and other simple derivatives may be called “reagents.” The starting material may be, for example, a pre-made reactant that can be purchased from suppliers or produced directly in-house. The “precursor” may correspond to a material at a stage before becoming a target material (or the target molecule G) that is finally obtained in a certain metabolism or chemical reaction.

As shown in the part 201, potential synthetic paths for synthesizing the target molecule 230, that is, synthetic path candidates may exist in various ways. The prediction apparatus according to an example embodiment may propose an optimal synthetic path to obtain the target molecule 230 with a highest yield among the potential synthetic paths using a pre-trained predictive model.

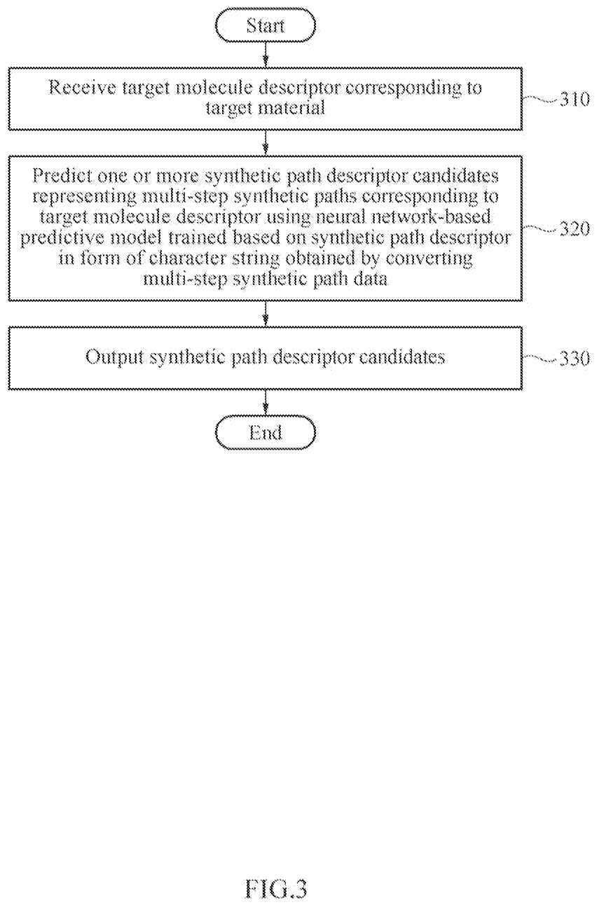

FIG. 3 is a flowchart illustrating a method of predicting a synthetic path according to an example embodiment. In the following example embodiments, operations may be sequentially performed, but are not necessarily performed sequentially. For example, the order of each operation may be changed, and at least two operations may be performed in parallel.

Referring to FIG. 3, a prediction apparatus according to an example embodiment may output synthetic path descriptor candidates through operations 310 through 330. The prediction apparatus according to an example embodiment may be implemented with various types of devices such as a personal computer (PC), a server device, a mobile device, and an embedded device, and for example, may correspond to a smartphone, a tablet device, an augmented reality (AR) device, an Internet of things (IoT) device, and/or a medical device that performs voice recognition, image recognition, and image classification using a neural network, but is not limited thereto. Further, the prediction apparatus may correspond to a dedicated hardware accelerator (HW accelerator) mounted on the above-described device, or may be a hardware accelerator such as a neural processing unit (NPU), a tensor processing unit (TPU), or a neural engine, which is a dedicated module for running a neural network, but is not limited thereto.

In operation 310, the prediction apparatus may receive a target molecule descriptor corresponding to a target material. The target molecule descriptor is a descriptor indicating molecular structure information corresponding to the target material, and may correspond to a molecular structure descriptor of a synthetic path descriptor. For example, when the target material is N-(4-phenylphenyl)pyridin-3-amine

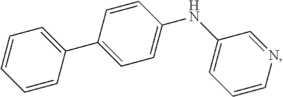 [SMILES: c1ccc(-c2ccc(Nc3cccnc3)cc2)cc1]

the target molecule descriptor may be a character string such as “c1ccc(−c2ccc(Nc3cccnc3)cc2)cc1” indicating molecular structure information including the types of atoms constituting the target material and a bonding structure between atoms but is not limited thereto.

In operation 320, the prediction apparatus may predict one or more synthetic path descriptor candidates representing multi-step synthetic paths corresponding to the target molecule descriptor input in operation 310, using a neural network-based predictive model trained based on a synthetic path descriptor in a form of a character string obtained by converting multi-step synthetic path data. The synthetic path descriptor will be described in detail with reference to FIGS. 4 and 5.

For example, a predictive model may predict the multi-step synthetic path descriptor in units of tokens based on the target molecule descriptor received in units of tokens. The predictive model may include, for example, an encoder and a decoder previously trained using the synthetic path descriptor in which a portion of tokens are masked. The predictive model may include, for example, a first encoder that extracts embedding information from a target molecule descriptor X, a second encoder previously trained to extract a first feature from the embedding information and a sequence including first tokens of a portion among tokens constituting the synthetic path descriptor, and a decoder previously trained to restore a character string corresponding to the synthetic path descriptor based on the first feature and information on second tokens other than the first tokens among the tokens constituting the synthetic path descriptor. The predictive model may be trained using a weight based on a difference between the synthetic path descriptor and the character string. The weight may be applied to and learned by at least one of the first encoder, the pre-trained second encoder, and/or the pre-trained decoder. A process of training the predictive model in advance will be described in detail with reference to FIGS. 6 and 7.

The predictive model may be, for example, a neural network in a tied two-way transformer structure. The predictive model may include, for example, a first predictive model that receives a first target molecule descriptor corresponding to the target material and predicts a synthetic path descriptor corresponding to the target material and a second predictive model that receives the synthetic path descriptor and predicts a second target molecule descriptor corresponding to the target material. A method in which the prediction apparatus predicts the synthetic path descriptor candidates using the predictive model will be described in detail with reference to FIGS. 8 and 9.

A method of predicting one or more synthetic path descriptor candidates by the prediction apparatus 100 and configurations and operations of predictive models for predicting the synthetic path descriptor candidates will be described in detail with reference to FIGS. 10 and 11.

The prediction apparatus may, for example, receive multi-step synthetic path data corresponding to various chemical materials, convert the multi-step synthetic path data into a synthetic path descriptor in a form of a character string, and store the synthetic path descriptor in a memory or database.

In operation 330, the prediction apparatus may output the synthetic path descriptor candidates predicted in operation 320. The prediction apparatus may aggregate tokens of the synthetic path descriptor predicted by the predictive model into one sequence and output the sequence as the synthetic path descriptor candidates.

FIG. 4 is a diagram illustrating a multi-step synthetic path descriptor according to an example embodiment. FIG. 4 illustrates a part 400 showing a synthetic path descriptor corresponding to synthetic paths for generating a target molecule according to an example embodiment. A part 410 shows synthetic paths for generating the target molecule through a multi-step synthesis process of reactants. A part 430 shows a synthetic path descriptor corresponding to the synthetic paths.

For example, as shown in the part 410, the multi-step synthesis process for generating a target molecule G may be performed through three steps including a first step (A+B→C), a second step (C+D→F), and a third step (E+F→G). In particular, reactants A, B, D, and E may correspond to starting materials to be purchased from, for example, chemical suppliers, an intermediate C may correspond to a reaction product (or intermediate product) generated in the first step, and an intermediate F may correspond to a reaction product generated in the second step.

As shown in the part 430, for example, the multi-step synthetic path descriptor may include tokens such as a molecular structure descriptor 431 representing molecular structure information of reactants, a notation character 433 for distinguishing synthesis steps before and after synthesis of the multi-step synthetic paths, a delimiting parenthesis 435 for defining a synthesis order of the reactants used for each synthesis step of the multi-step synthetic paths, and a separator 437 for distinguishing between the molecular structure descriptor 431 and the delimiting parenthesis 435 or distinguishing between a plurality of molecular structure descriptors 431 A “token” may correspond to a character, a number, and/or a symbol representing each component (e.g., a molecular structure descriptor, a notation character, a delimiting parenthesis, a separator, and/or a reaction descriptor corresponding to a reaction method) of the synthetic path descriptor but is not limited thereto.

The molecular structure descriptor 431 may correspond to a one-dimensional ASCII character array representing structural information of a single molecule. Here, the structural information may correspond to a structural formula based on a bond between atoms. The molecular structure descriptor 431 may be, for example, a string descriptor such as a Simplified Molecular-Input Line-Entry System (SMILES) code, a Smiles Arbitrary Target Specification (SMARTS) code, and/or an International Chemical Identifier (InChi) code but is not limited thereto.

The molecular structure descriptor 431 may include tokens representing at least one of, for example, types of atoms constituting each of reactants A, B, D, and E and intermediates C and F, bonding information including a bonding structure between the atoms, an aromatic compound corresponding to molecular structure information, and an isomer corresponding to the molecular structure information.

Here, the “aromatic compound” may correspond to a compound in which pi (it) electrons are delocalized by alternately connecting single bonds and double bonds to form a ring. The aromatic compound may be classified into, for example, a compound having a hydrocarbon and a substituent, and a heterocyclic compound. The “hydrocarbon” may correspond to an organic compound consisting of only carbon (C) and hydrogen (H). A “substituent” may correspond to, for example, an atom or group of atoms replacing one or more hydrogen atoms (R1) on a parent chain of the hydrocarbon. For example, chlorobenzene (C₆H₅Cₗ) may be obtained by substituting one hydrogen (H) atom with a chlorine (Cl) atom in benzene (C₆H₆), and nitrobenzene (C₆H₅NO₂) may be obtained by substituting one hydrogen (H) atom of benzene with a nitro group (NO₂). In this example, the chlorine atom and the nitro group, each substituted with one hydrogen atom may correspond to the substituent. In addition, the “heterocyclic compound” may correspond to, for example, a compound having two or more elements constituting a ring among ring compounds such as

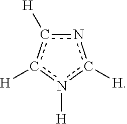

The “isomer” may correspond to a compound having the same molecular structural formula but not the same connection method or spatial arrangement of members in a molecule. The isomer may include, for example, a structural isomer, a geometric isomer, and/or an optical isomer.

The notation character 433 that distinguishes the synthesis step may be, for example, “>>”, but is not limited thereto. Based on the notation character 433 “>>”, the left may indicate before performing the synthesis of each step and the right may indicate after performing the synthesis.

The delimiting parenthesis 435 may define a synthesis order of reactants used for each synthesis step of multi-step synthetic paths. The delimiting parenthesis 435 may be, for example, a pair of “{” and “}” but is not limited thereto. Descriptors enclosed in innermost parentheses (e.g., A and B descriptors) may correspond to the synthesis reactants of the corresponding step (e.g., the reactant C).

The separator 437 may correspond to a character that distinguishes between a single molecular structure descriptor and a delimiting parenthesis, or distinguishes between a plurality of molecular structure descriptors, that is, one molecular structure descriptor and another molecular structure descriptor. The separator may be, for example, “.” but is not limited thereto.

In the part 430, based on the notation character 433 “>>”, a leftmost character string “{E descriptor. {{A descriptor. B descriptor}. D descriptor}}” may indicate a situation before a first-step synthesis. Here, molecules A and B of “{A descriptor. B descriptor}” enclosed in the innermost delimiting parenthesis 435 may correspond to first-step synthesis reactants. By replacing a character string “{A descriptor. B descriptor}” with a “descriptor C” for a synthesis product C, a situation after the first-step synthesis may be expressed as “{E descriptor. {C descriptor. D descriptor}}.” The entire synthesis path may be expressed by applying such process to a second-step synthesis and a third-step synthesis.

In contrast, the multi-step synthetic descriptor shown in the part 430 may be restored as a diagram such as the part 410.

In an example embodiment, by using the synthetic path descriptor in the form of the character string, a process of synthesizing a compound by a neural network-based predictive model may be grasped readably for each step. In addition, by allowing the neural network-based predictive model to learn each step along with the synthetic path descriptor, a target material synthetic path used for synthesis for each step may be predicted quickly and accurately.

FIG. 5 is a diagram illustrating a multi-step synthetic path descriptor according to another example embodiment. FIG. 5 illustrates a part 500 which shows a synthetic path descriptor corresponding to synthetic paths for generating a target molecule according to an example embodiment. A part 510 shows synthetic paths for generating the target molecule through a multi-step synthesis process of reactants. A part 530 shows a synthetic path descriptor corresponding to the synthetic paths.

In some example embodiments, the synthetic path descriptor may further include a reaction descriptor 535 corresponding to a reaction method used for each of the multi-step synthetic paths in addition to the molecular structure descriptor 431, the notation character 433, the delimiting parenthesis 435, and the separator 437 described above.

In a case of adopting various reaction methods in addition to structural information of the reactants, a yield at the time of synthesis may be improved. In an example embodiment, by allowing a predictive model to predict not only the structure of the reactant but also the synthesis method for each path, that is, the reaction method, it is possible to infer the yield in addition to a relevant synthesis condition. Since the reaction method is information that may contain elements necessary for most synthetic experiments, the predictive model may be extended by learning along with the structure of the reactant.

The “reaction method” may correspond to a chemical reaction method for generating a product using synthesized reactants. The reaction method may include, for example, a Suzuki-miyaura reaction method, a Buchwald reaction method, and/or an arylation reaction method but is not limited thereto. For example, in a case in which a target material halide (R2-BY2) is generated using a reactant organic boron (R1-BY2), the reaction method may be the Suzuki-miyaura reaction method. A plurality of reaction methods may be provided based on, for example, reactant structural information and product structural information (e.g., A molecular structure+B molecular structure=C molecular structure). Here, the “structure” may refer to an atom-level structure of a material.

For example, as shown in the part 530, when a synthesis of reactant A and reactant B is performed in a first step using the Suzuki-miyaura reaction method, “S” which is the descriptor 535 corresponding to the reaction method (e.g., the Suzuki-miyaura reaction method) may be added between the notation character (“>>”) indicating a synthesis step so as to be read as “>S>”. In addition, when the Buchwald reaction method is used to perform a synthesis of intermediate C and reactant D in a second step and a synthesis of reactant E and intermediate F in a third step, “B” which is the descriptor 535 corresponding to the reaction method (e.g., the Buchwald reaction method) may be added between the notation character (“>>”) indicating a synthesis step so as to be read as “>B>”.

FIG. 6 is a diagram illustrating a process of training a predictive model in advance according to an example embodiment. FIG. 6 illustrates an example 600 of a pre-trained model 601 used in a process of training a predictive model in advance and a fine-tuning model 603 that fine-tunes a result predicted by the pre-trained model 601 according to an example embodiment.

For example, a multi-step synthetic path descriptor such as “{A. B}>>C” may be input to the pre-trained model 601. In particular, the pre-trained model 601 may output the multi-step synthetic path descriptor “{A. B}>>C” predicted through an encoder 610 and a decoder 620. The encoder 610 and the decoder 620 trained in the pre-trained model 601 may be used as an encoder 640 and a decoder 650 of the fine-tuning model 603.

The fine-tuning model 603 may further include an encoder 630 in addition to the encoder 640 and the decoder 650, and the fine-tuning model 603 may be used as a predictive model for predicting a synthetic path candidate.

A predictive model according to an example embodiment may receive a target molecule descriptor corresponding to a target molecule as an input and predict one or more synthetic path descriptor candidates representing multi-step synthetic paths corresponding to the target molecule descriptor. Accordingly, the fine-tuning model 603 may be trained to receive a target molecule descriptor C as an input and output a multi-step synthetic path descriptor “{A. B}” corresponding to the target molecule descriptor C.

In an example embodiment, by learning a synthetic context that is difficult to be extracted from a single-step descriptor, a predictive model may predict a multi-step synthetic path with high accuracy. In addition, since the prediction model proposes multi-step synthetic paths as a character string at a time without using a separate retrieval algorithm, the prediction may be performed with increased speed. A process of training the pre-trained model 601 and the fine-tuning model 603 will be described in detail with reference to FIG. 7.

FIG. 7 is a diagram illustrating a method of training a predictive model according to an example embodiment. FIG. 7 illustrates an example 700 of a process of training a predictive model for predicting a multi-step synthetic path R_{pred} to synthesize a target molecule descriptor X by a training apparatus according to an example embodiment.

In operation 710, a training apparatus may receive multi-step synthetic path data corresponding to chemical materials. Synthetic path data may be, for example, data corresponding to multi-step synthetic paths extracted from a database but is not limited thereto.

In operation 720, the training apparatus may convert the multi-step synthetic path data received in operation 710 into a multi-step synthetic path descriptor in a form of a character string. For example, the training apparatus may distinguish between a synthetic path descriptor 721 used in a pre-training process of a pre-trained model 740 and a synthetic path descriptor 723 used in a process of training a fine-tuning model 760 and convert the synthetic path descriptor 721 and the synthetic path descriptor 723.

The synthetic path descriptor 721 may be, for example, a synthetic path descriptor corresponding to all intermediate reactants (or intermediates) in addition to a starting material. The synthetic path descriptor 723 may be, for example, a synthetic path descriptor corresponding to a starting material.

The training apparatus may classify materials used for each neural network model (e.g., the pre-trained model 740 and the fine-tuning model 760) based on the methods described with reference to FIGS. 4 and 5 and perform a conversion into a multi-step synthetic path descriptor.

A predictive model according to an example embodiment may be trained to predict the multi-step synthetic path R_{pred} by receiving the entire character string of a given target molecule descriptor X. The predictive model may be trained through, for example, the pre-training process by the pre-trained model 740 and a fine-tuning process by the fine-tuning model 760. In general, it is known that a model with higher performance is obtained by performing the pre-training as compared to training performed by applying the fine-tuning.

In operation 730, the training apparatus may train the pre-trained model 740. For example, the training apparatus may obtain the pre-trained model 740 including an encoder 741 and a decoder 743 and data on a descriptor 721 for pre-training. The pre-trained model 740 may be constructed using various language model structures in an encoder-decoder format. The pre-trained model 740 may be constructed by, for example, a transformer language model structure but is not limited thereto.

The training apparatus according to an example embodiment may randomly select about 80% of all tokens included in the multi-step synthetic path descriptor as tokens to be masked when training the pre-trained model 740, but it is merely an example. The training apparatus may select tokens to be masked from all tokens of the multi-step synthetic path descriptor, for example, in units of molecule smiles, in units of delimiting parentheses for defining a synthesis order (or synthesis step), and/or in units of molecular structure descriptors.

The encoder 741 of the pre-trained model 740 may receive a sequence in which a portion of tokens in a multi-step synthetic path descriptor R are masked. The encoder 741 may extract the first feature from a sequence in which tokens of the portion among the tokens constituting the synthetic path descriptor are randomly masked, extract the first feature from a sequence in which the tokens constituting the synthetic path descriptor are masked in units of delimiting parentheses for defining a synthesis order, or extract the first feature from a sequence in which the tokens constituting the synthetic path descriptor are masked in molecular structure descriptor units. The encoder 741 may correspond to, for example, a retrosynthesis encoder 1123 of FIG. 11.

The decoder 743 of the pre-trained model 740 may receive information on unmasked surrounding tokens in the multi-step synthetic path descriptor R, for example, surrounding context information. The decoder 743 may restore a full character string of the multi-step synthetic path descriptor R_{pred} using an output of the encoder 741 and the information on the unmasked surrounding tokens in the multi-step synthetic path descriptor R. For example, the decoder 743 may predict tokens in sequence such as “token1→token2→token3→ . . . ”, starting from a token <sos> indicating the beginning of the character string, and when a token <eos> indicating the end of the character string is predicted, suspend decoding. One or more weights (and biases) of the predictive model may be updated to minimize or converge, for example, a value (e.g., loss) of ∥R−R_{pred}∥. The loss of the predictive model may be determined to be minimized or converge when the loss has reached a predetermined threshold value or its local minimum value, or when the loss does not reduce any longer through an iteration process and therefore has reached a constant value (with a predetermined margin). The decoder 743 may correspond to, for example, a retrosynthesis decoder 1124 of FIG. 11.

In operation 750, the training apparatus may train the fine-tuning model 760. For example, the fine-tuning model 760 may be trained to predict a candidate for the multi-step synthetic path descriptor R_{pred} corresponding to a synthetic path of the given target molecule descriptor X.

For example, a multi-step synthetic path descriptor Ram which is a target for training in the process of training the fine-tuning model 760 may be synthetic data of which all of the starting materials are purchasable. In contrast, in the process of training the pre-trained model 740, since the corresponding data may be used as training data even when not all of the starting materials are purchasable, a larger quantity of data may be used for training compared to the fine-tuning model 760.

The fine-tuning model 760 may be trained to predict the multi-step synthetic path descriptor in units of tokens based on the target molecule descriptor received in units of tokens. Accordingly, all target molecule descriptors may be tokenized in character units representing atom types and/or chemical bonds before being processed in the fine-tuning model 760.

The “character units” representing atom types and/or chemical bonds may include, for example, a token (e.g., <sos>) indicating the beginning of the character string (e.g., start of sequence) and a token (e.g., <eos>) indicating the end of the character string (e.g., end of sequence) in addition to alphabet letters, numbers, shapes, and symbols representing types of atoms corresponding to molecular structure information, bonding information including a bonding structure between the atoms, an aromatic compound corresponding to the molecular structure information, and an isomer corresponding to the molecular structure information. For example, when a target molecule descriptor is “c1ccc(−c2ccc(Nc3cccnc3)cc2)cc1”, each character unit (e.g., ‘c’, ‘l’, ‘c’, ‘c’, ‘c’, ‘(’, ‘−’, ‘c’, ‘2’, . . . , ‘)’, ‘c’, ‘c’, and ‘1’) included in the target molecule descriptor may correspond to a token. In addition, each component (e.g., a molecular structure descriptor, a notation character, a delimiting parenthesis, a separator, and/or a reaction descriptor corresponding to a reaction method) of the synthetic path descriptor may be a token. Likewise, all target molecule descriptors may be tokenized before being processed by a predictive model so as to be input to the predictive model.

The training apparatus may aggregate the tokens predicted by the fine-tuning model 760 into one sequence and output the sequence as a multi-step synthetic path descriptor candidate.

The fine-tuning model 760 may also use a language model structure of an encoder-decoder format similarly to the pre-trained model 740. An encoder 763 and a decoder 765 included in the fine-tuning model 760 may correspond to the encoder 741 and the decoder 743 trained in operation 730. Since the target molecule descriptor X is input to the fine-tuning model 760 instead of the multi-step synthetic path descriptor R in the fine-tuning process of operation 750, a new encoder 761 that encodes the target molecule descriptor X in a form of the multi-step synthetic path descriptor R may be added to an end in front of the encoder 741 or 763 and the decoder 743 or 765 trained by the pre-trained model 740 in operation 730, so as to be trained.

The training apparatus may update the fine-tuning model 760 to minimize or converge a difference value (e.g., ∥Rₐₙₛ−R_{pred}∥) between the multi-step synthetic path descriptor Rₐₙₛ that is a ground truth to be learned in the fine-tuning process of operation 750 and the multi-step synthetic path descriptor R_{pred} predicted by the fine-tuning model 760. The difference value may indicate a loss of the fine-tuning model 760.

In particular, the fine-tuning model 760 may be adjusted in various ways according to a method of updating one or more weights (and biases) of the fine-tuning model 760 by back-propagating the difference value (e.g., ∥Rₐₙₛ−R_{pred}∥). For example, the training apparatus may update the weight of the newly added encoder 761, update a portion of weights of the pre-trained encoder 763 and the pre-trained decoder 765, or update the weights of the encoder 761, the pre-trained encoder 763, and the pre-trained decoder 765.

The prediction apparatus may predict the synthetic path descriptor candidates using the fine-tuning model 760 trained through the above-described process as the predictive model.

The fine-tuning model 760 may also be trained by, for example, a tied two-way transformer structure. The fine-tuning model 760 trained by the tied two-way transformer structure may output the multi-step synthetic path descriptor R_{pred} predicted by receiving the target molecule descriptor X and may also output a target molecule descriptor X_{pred} predicted by receiving the multi-step synthetic path descriptor R. An operation performed in a case in which the fine-tuning model 760 is trained based on the tied two-way transformer structure will be described in detail with reference to FIG. 8.

FIG. 8 is a diagram illustrating a process of predicting a synthetic path using a trained predictive model according to an example embodiment. FIG. 8 illustrates an example 800 of a process of predicting a multi-step synthetic path candidate from a target molecule descriptor corresponding to a target material according to an example embodiment.

In operation 810, a prediction apparatus may receive a target molecule descriptor 815 corresponding to a target molecule of which a synthetic path is to be acquired.

In operation 820, the prediction apparatus may apply the target molecule descriptor 815 to the first predictive model 825. In particular, the first predictive model 825 may correspond to the fine-tuning model trained through the aforementioned operation 750. The first predictive model 825 may include, for example, an encoder 1, a pre-trained encoder 1, and a pre-trained decoder 1.

In operation 830, the prediction apparatus may acquire a multi-step synthetic path descriptor 835 in a form of a token predicted by the first predictive model 825.

In operation 840, the prediction apparatus may determine whether a candidate material corresponding to a synthetic path descriptor obtained by aggregating the multi-step synthetic path descriptor 835 in the form of the token acquired in operation 830 is a starting material of which a chemical characteristic and a structure are known.

When it is determined in operation 840 that the candidate material is not the starting material, in operation 850, the prediction apparatus may remove the synthetic path descriptor acquired in operation 830 from synthetic path descriptor candidates.

In contrast, when it is determined in operation 840 that the candidate material is the starting material, in operation 860, the prediction apparatus may apply the multi-step synthetic path descriptor 835 predicted in operation 830 to the second predictive model 865. The second predictive model 865 may have the same or substantially the same components as the first predictive model 825 and have an input and output inversed from those of the first predictive model 825. In operation 870, the prediction apparatus may acquire the target molecule descriptor 875 predicted by the second predictive model 865 to correspond to the multi-step synthetic path descriptor 835 by applying the multi-step synthetic path descriptor 835 to the second predictive model 865. The second predictive model 865 may include, for example, an encoder 2, a pre-trained encoder 2, and a pre-trained decoder 2.

In terms of the encoder 1, the pre-trained encoder 1, and the pre-trained decoder 1 included in the first predictive model 825 and the encoder 2, the pre-trained encoder 2, and the pre-trained decoder 2 included in the second predictive model 865, components and operations may be the same and inputs and outputs may be inversed.

In operation 880, the prediction apparatus may determine whether the target molecule descriptor 815 received in operation 810 matches the target molecule descriptor 875 predicted in operation 870.

When it is determined in operation 880 that the target molecule descriptor 815 matches the target molecule descriptor 875, in operation 890, the prediction apparatus may determine the multi-step synthetic path descriptor predicted in operation 830 to be a multi-step synthetic path candidate 895.

In contrast, when it is determined in operation 880 that the target molecule descriptor 815 does not match the target molecule descriptor 875, the prediction apparatus may remove the multi-step synthetic path descriptor 835 predicted in operation 830 from the synthetic path descriptor candidates through operation 850.

FIG. 9 is a flowchart illustrating a method of predicting one or more synthetic path descriptor candidates according to an example embodiment. In the following example embodiments, operations may be sequentially performed, but are not necessarily performed sequentially. For example, the order of each operation may be changed, and at least two operations may be performed in parallel.

Referring to FIG. 9, a prediction apparatus according to an example embodiment may predict synthetic path descriptor candidates through operations 910 through 960.

In operation 910, the prediction apparatus may apply a first target molecule descriptor to a first predictive model. The prediction apparatus may classify one or more characters representing an atom type of a target material and one or more characters representing a chemical bond of the target material in the first target molecule descriptor as tokens. The prediction apparatus may apply at least a portion of the classified tokens to the first predictive model.

In operation 920, the prediction apparatus may acquire a synthetic path descriptor predicted by the first predictive model based on the first target molecule descriptor.

In operation 930, the prediction apparatus may determine whether a candidate material corresponding to the synthetic path descriptor predicted in operation 920 is a starting material of which a chemical characteristic and a structure are known. For example, the prediction apparatus may determine whether the candidate material is the starting material using data registered in a material database.

When it is determined in operation 930 that the candidate material is not the starting material, in operation 940, the prediction apparatus may remove a synthetic path descriptor corresponding to the candidate material from the synthetic path descriptor candidates.

When it is determined in operation 930 that the candidate material is the starting material, in operation 950, the prediction apparatus may predict a second target molecule descriptor by applying the synthetic path descriptor predicted in operation 920 to a second predictive model.

In operation 960, the prediction apparatus may predict one or more synthetic path descriptor candidates based on whether the first target molecule descriptor matches the second target molecule descriptor. The prediction apparatus may provide a synthesis recipe corresponding to the target material based on the synthetic path descriptor candidates. A method in which the prediction apparatus predicts one or more synthetic path descriptor candidates by comparing the first target molecule descriptor and the second target molecule descriptor will be described in detail with reference to FIG. 10.

FIG. 10 is a diagram illustrating a method of predicting one or more synthetic path descriptor candidates by a prediction apparatus according to an example embodiment.

FIG. 10 illustrates an example 1000 for explaining a method of predicting one or more synthetic path descriptor candidates by a prediction apparatus (e.g., the prediction apparatus 100 of FIG. 1) according to an example embodiment. The prediction apparatus may receive a target molecule descriptor 1010 obtained by expressing a chemical structure of a target molecule in a form of a character string 1011 such as “c1ccc(−c2ccc(Nc3cccnc3)cc2)cc1”, for example.

The prediction apparatus may input the target molecule descriptor 1010 to the first predictive model 825. The latent variable 1020 may also be input to the first predictive model 825. A “latent variable” 1020 may correspond to a variable affecting information on a multi-step synthetic path descriptor 1030, which is not observed directly. In an example embodiment, the latent variable 1020 may indicate, for example, a type of reaction. The type of reaction may include a reaction type such as decomposition, combustion, metathesis, and displacement, experimental conditions such as catalyst, base, solvent, reagent, temperature, and reaction time, and/or a reaction method such as Suzuki-miyaura. In addition, the latent variable 1020 may include a plurality of classes. The plurality of classes may be generated to correspond to the type of reaction. A cluster of synthetic path combinations may be changed by the latent variable 1020.

The first predictive model 825 may be, for example, a retrosynthesis predictive model that is a probability model depending on the latent variable 1020 which is not observed. The retrosynthesis predictive model may be a Gaussian mixture model including a plurality of normal distributions in which information on synthetic path combinations or synthetic paths has different parameters (for example, an expected value, a fractional phase, etc.) based on the latent variable 1020 but is not limited thereto.

The prediction apparatus may output the multi-step synthetic path descriptor 1030 for generating the target molecule descriptor 1010 using the first predictive model 825 trained in advance. The multi-step synthetic path descriptor 1030 may be output in a form of a character string in units of tokens. In other words, the prediction apparatus may output the multi-step synthetic path descriptor 1030 in the form of the character string based on the latent variable 1020 and the target molecule descriptor 1011 in the form of the character string. In an example embodiment, the prediction apparatus may output the multi-step synthetic path descriptor 1030 that maximizes a likelihood for each class with respect to an input of the target molecule descriptor 1010 and a likelihood of a retrosynthesis prediction result but is not limited thereto.

The multi-step synthetic path descriptor 1030 may correspond to a set of multi-step synthetic path candidates predicted by the prediction apparatus to generate the target molecule descriptor 1010. The set of multi-step synthetic path candidates may include a single synthetic path candidate or a plurality of synthetic path candidates.

Meanwhile, when the latent variable 1020 is input to the first predictive model 825, a diversity for the multi-step synthetic path descriptor 1030 may be secured.

The prediction apparatus may input the multi-step synthetic path descriptor 1030 to the second predictive model 865. The latent variable 1020 may also be input to the second predictive model 865.

The second predictive model 865 may correspond to a path prediction model.

The prediction apparatus may output a target molecule descriptor 1050 predicted by the second predictive model 865 to correspond to the multi-step synthetic path descriptor 1030 using the second predictive model 865 trained in advance. The predicted target molecule descriptor 1050 may be output in the form of the character string. In other words, the prediction apparatus may output the predicted target molecule descriptor 1050 in the form of the character string based on the latent variable 1020 and the multi-step synthetic path descriptor 1030 in the form of the character string.

The predicted target molecule descriptor 1050 may correspond to a molecular structure descriptor of a product synthesized through synthetic paths included in the multi-step synthetic path descriptor 1030.

Meanwhile, since the latent variable 1020 is input to the second predictive model 865, a diversity of the predicted target molecule descriptor 1050 may be secured.

The prediction apparatus may compare the input target molecule descriptor 1010 and the target molecule descriptor 1050 predicted for each input of the multi-step synthetic path descriptor 1030 as indicated by reference numeral 1060. In addition, the prediction apparatus may determine a priority of the multi-step synthetic path descriptor 1030 based on a result of the comparison of the reference numeral 1060. The prediction apparatus may output a multi-step synthetic path candidate 1070 based on the priority of the multi-step synthetic path descriptor 1030.

The prediction apparatus according to an example embodiment may verify the multi-step synthetic path descriptor 1030 derived through the first predictive model 825 using the second predictive model 865. Accordingly, the prediction apparatus may prevent a case in which the multi-step synthetic path descriptor 1030 that is against a structural grammar is output and/or a case in which the target molecule descriptor 1010 is not to be synthesized in the form of the character string.

FIG. 11 is a diagram illustrating operations computed in a first predictive model and a second predictive model according to an example embodiment. FIG. 11 illustrates an example 1100 for explaining configurations of the first predictive model 825 and the second predictive model 865 and operations computed in the first predictive model 825 and the second predictive model 865 according to an example embodiment.

A prediction apparatus may receive a target molecule descriptor X 1110 represented in a form of a character string. The prediction apparatus may input the target molecule descriptor X 1110 in the form of the character string to the first predictive model 825.

The prediction apparatus may predict the latent variable 1020 based on the target molecule descriptor X 1110. The latent variable 1020 may indicate, for example, variables affecting information on a multi-step synthetic path descriptor 1130, which is not directly observed, and may include a plurality of classes.

The prediction apparatus may predict the multi-step synthetic path descriptor 1130 for generating the target molecule descriptor X 1110 by running the first predictive model 825 based on the target molecule descriptor X 1110 and the latent variable 1020.

The first predictive model 825 may include, for example, a word embedder 1121, position encoders 1122a, 1122b, 1122c, and 1122d (hereinafter, indicated as a position encoder 1122 when there is no need for distinction), a retrosynthesis encoder 1123, a retrosynthesis decoder 1124, and a word generator 1125.

Although FIG. 11 illustrates the word embedder 1121, the position encoder 1122, the retrosynthesis encoder 1123, the retrosynthesis decoder 1124, and the word generator 1125 as units included in the first predictive model 825, the word embedder 1121, the position encoder 1122, the retrosynthesis encoder 1123, the retrosynthesis decoder 1124, and the word generator 1125 may also be layers included in the first predictive model 825.

The word embedder 1121 may embed input data by character. The word embedder 1121 may map the target molecule descriptor X 1110 expressed in a form of a character string to a preset dimensional vector. In addition, the word embedder 1121 may map the latent variable 1020 to a preset dimensional vector.

The position encoder 1122 may perform positional encoding to identify positions of characters included in the input data (e.g., the target molecule descriptor X 1110 expressed in a form of a character string). In an example embodiment, the position encoder 1122 may encode input data using sine waves of different frequencies, but is not limited thereto.

A first position encoder, for example, the position encoder 1122a may perform the positional encoding on the target molecule descriptor X 1110 in the form of the character string. The first position encoder 1122a may combine the embedded input data (e.g., the target molecule descriptor X 1110 embedded by character) with positional information and provide a result of the combination to the retrosynthesis encoder 1123.

A second position encoder, for example, the position encoder 1122b may perform the positional encoding on the latent variable 1020 as an initial token. The second position encoder 1122b may combine the embedded input data (e.g., the latent variable 1020) and positional information and provide a result of the combination to the retrosynthesis encoder 1123.

The retrosynthesis encoder 1123 may include, for example, a self-attention sublayer and a feed-forward sublayer. Although FIG. 11 illustrates the retrosynthesis encoder 1123 as a single unit for ease of description, in some cases, the retrosynthesis encoder 1123 may be configured in a form in which N encoders are layered.

The retrosynthesis encoder 1123 may specify information to be paid attention to from an input sequence of the target molecule descriptor X 1110 through the self-attention sublayer. The specified information may be transferred to the feed-forward sublayer. The feed-forward sublayer may include a feed-forward neural network. Through the feed-forward neural network, a transformed sequence of the input sequence may be output. The transformed sequence may be provided to the retrosynthesis decoder 1124 as an encoder output of the retrosynthesis encoder 1123.

Like the retrosynthesis encoder 1123, FIG. 11 illustrates the retrosynthesis decoder 1124 as a single unit for ease of description. However, in some cases, the retrosynthesis decoder 1124 may be configured in a form in which N decoders are layered.

The retrosynthesis decoder 1124 may include, for example, the self-attention sublayer, an encoder-decoder attention sublayer, and the feed-forward sublayer. The encoder-decoder attention sublayer may differ from the self-attention sublayer in that a query is a vector of a decoder and a key and a value are vectors of an encoder.

Meanwhile, a residual connection sublayer and a normalization sublayer may be applied to all subordinate layers individually, and masking may be applied to the self-attention sublayer to prevent a current output position from being used as information on a subsequent output position. In addition, an output of the retrosynthesis decoder 1124 may be linearly converted or a softmax function may be applied.

The retrosynthesis decoder 1124 may output sequences corresponding to an input sequence of the latent variable 1020 and an input sequence of the target molecule descriptor X 1110 using a beam search procedure. The output sequence may be converted into a form of a character string by the word generator 1125 and then output. Through this, the first predictive model 825 may output the multi-step synthetic path descriptor 1130 in a form of a character string corresponding to the input of the target molecule descriptor X 1110. In addition, the latent variable 1020 may be shared with the second predictive model 865 and used for a computational operation of a target molecule descriptor 1150 predicted.

The prediction apparatus may run the second predictive model 865 based on the latent variable 1020 and the multi-step synthetic path descriptor 1130 output from the first predictive model 825, thereby predicting the target molecule descriptor 1150 predicted for the multi-step synthetic path descriptor 1130.

Like the first predictive model 825, the second predictive model 865 may include the word embedder 1121, the position encoder 1122, a reaction prediction encoder 1123, a retrosynthesis decoder 1124, and the word generator 1125. The retrosynthesis decoder 1124 may be referred to as a “reaction prediction decoder”.

Since the first predictive model 825 and the second predictive model 865 share the encoders 1121 and 1123, the retrosynthesis encoder 1123 of the first predictive model 825 and the retrosynthesis encoder 1123 of the second predictive model 865 may each be referred to as a “reaction prediction encoder”. The retrosynthesis encoder 1123 of the first predictive model 825 and the retrosynthesis encoder 1123 of the second predictive model 865 may be collectively referred to as a shared encoder 1123. In addition, although FIG. 11 illustrates each of the word embedder 1121, the position encoder 1122, the reaction prediction encoder 1123, the reaction prediction encoder 1124, and the word generator 1125 as a single unit included in the second predictive model 865, the word embedder 1121, the position encoder 1122, the reaction prediction encoder 1123, the reaction prediction encoder 1124, and the word generator 1125 may each be a layer included in the second predictive model 865.

A computation method of the second predictive model 865 and a computation method of the first predictive model 825 may be similar to each other except for a type of an input sequence and a type of an output sequence. In other words, the word embedder 1121 may receive the multi-step synthetic path descriptor 1130 and the latent variable 1020 as input data and embed the input data by character. In addition, a third position encoder, for example, the position encoder 1122c of the second predictive model 865 may combine the embedded input data and positional information and provide a result of the combination to the reaction prediction encoder 1123. Also, a fourth position encoder, for example, the position encoder 1122d of the second predictive model 865 may perform positional encoding on the latent variable 1020 as an initial token and provide a result of the positional encoding to the reaction prediction encoder 1124.

The reaction prediction encoder 1123 may specify information to be paid attention to from an input sequence of the multi-step synthetic path descriptor 1130 through the self-attention sublayer and transfer the specified information to the feed-forward sublayer. The feed-forward sublayer may output a transformed sequence using the feed-forward neural network.

The reaction prediction encoder 1124 may include the self-attention sublayer, the encoder-decoder attention sublayer, and the feed-forward sublayer. In addition, the residual connection sublayer and the normalization sublayer may be applied to all subordinate layers individually, and masking may be applied to the self-attention sublayer to prevent a current output position from being used as information on a subsequent output position. Also, a decoder output may be linearly converted or a softmax function may be applied.

The reaction prediction encoder 1124 may output sequences corresponding to the input sequence of the latent variable 1020 and the input sequence of the multi-step synthetic path descriptor 1130 using the beam search procedure. The output sequence may be converted into a form of a character string by the word generator 1125 and then output. Through this, the second predictive model 865 may output the predicted target molecule descriptor 1150 in the form of the character string corresponding to the input of the multi-step synthetic path descriptor 1130.

The prediction apparatus may compare the target molecule descriptor 1011 in the form of the character string and the predicted target molecule descriptor 1150 in the form of the character string as indicated by reference numeral 1160 and determine multi-step synthetic path candidates 1170 based on a comparison result. The prediction apparatus may determine priorities in the multi-step synthetic path descriptor 1130 based on whether the target molecule descriptor 1011 in the form of the character string matches the predicted target molecule descriptor 1150 in the form of the character string and determine the multi-step synthetic path candidates 1170 based on the priorities.

Meanwhile, since a source language and a target language are identically in a form of a character string (for example, SMILES) as shown in FIG. 11, the first predictive model 825 and the second predictive model 865 may share the word embedder 1121, the word generator 1125, and the encoder 1123. In addition, since the first predictive model 825 and the second predictive model 865 share the same parameter as the latent variable 1020, a model complexity may be reduced and a model regularization may be performed with increased ease.

The example embodiments described herein may be implemented using hardware components, software components, and/or a combination thereof. For example, the processing device and the component described herein may be implemented using one or more general-purpose or special purpose computers, such as, for example, a processor, a controller and an arithmetic logic unit (ALU), a digital signal processor, a microcomputer, a field programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will be appreciated that a processing device may include multiple processing elements and/or multiple types of processing elements. For example, a processing device may include multiple processors or a processor and a controller. In addition, different processing configurations are possible, such as parallel processors.

The software may include a computer program, a piece of code, an instruction, or some combination thereof, to independently or collectively instruct and/or configure the processing device to operate as desired, thereby transforming the processing device into a special purpose processor. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. The software and data may be stored by one or more non-transitory computer readable recording mediums.

The methods according to the above-described example embodiments may be recorded in non-transitory computer-readable media including program instructions to implement various operations of the above-described example embodiments. The media may also include, alone or in combination with the program instructions, data files, data structures, and the like. The program instructions recorded on the media may be those specially designed and constructed for the purposes of example embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher level code that may be executed by the computer using an interpreter.

The above-described hardware devices may be configured to act as one or more software modules in order to perform the operations of the above-described example embodiments, or vice versa.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A method of predicting a synthetic path, the method comprising:
receiving a target molecule descriptor corresponding to a target material;
predicting one or more synthetic path descriptor candidates representing multi-step synthetic paths corresponding to the target molecule descriptor using a neural network-based predictive model, the neural network-based predictive model being trained based on a synthetic path descriptor obtained by converting multi-step synthetic path data; and
outputting the one or more synthetic path descriptor candidates.

2. The method of claim 1, wherein the synthetic path descriptor comprises one or more tokens among:
a molecular structure descriptor representing molecular structure information of reactants;
a notation character for distinguishing synthesis steps before and after synthesis of the multi-step synthetic paths;
a delimiting parenthesis for defining a synthesis order of the reactants used for each synthesis step of the multi-step synthetic paths;
a separator for distinguishing between the molecular structure descriptor and the delimiting parenthesis or distinguishing between a plurality of molecular structure descriptors; and
a reaction descriptor corresponding to a reaction scheme used for each synthesis step of the multi-step synthetic paths.

3. The method of claim 2, wherein the molecular structure descriptor comprises a token representing at least one of types of atoms included in the reactants, bonding information comprising a bonding structure of the atoms, an aromatic compound corresponding to the molecular structure information, or an isomer corresponding to the molecular structure information.

4. The method of claim 3, wherein the neural network-based predictive model comprises an encoder and a decoder that are trained using the synthetic path descriptor in which a portion of tokens are masked.

5. The method of claim 3, wherein the neural network-based predictive model comprises:
a first encoder configured to extract embedding information from the target molecule descriptor;
a second encoder that is trained to extract a first feature from the embedding information and a sequence comprising first tokens of a portion among tokens constituting the synthetic path descriptor; and
a decoder that is trained to restore a character string corresponding to the synthetic path descriptor based on the first feature and information on second tokens other than the first tokens among the tokens constituting the synthetic path descriptor, and
wherein the neural network-based predictive model is trained by updating one or more weights of the predictive model based on a difference between the synthetic path descriptor and the character string.

6. The method of claim 5, wherein the second encoder is configured to:
extract the first feature from a first sequence in which tokens of the portion among the tokens constituting the synthetic path descriptor are randomly masked;
extract the first feature from a second sequence in which the tokens constituting the synthetic path descriptor are masked in units of delimiting parentheses for defining the synthesis order; or
extract the first feature from a third sequence in which the tokens constituting the synthetic path descriptor are masked in units of molecular structure descriptors.

7. The method of claim 1, wherein the neural network-based predictive model comprises:
a first predictive model configured to receive a first target molecule descriptor corresponding to the target material and predict the synthetic path descriptor corresponding to the target material; and
a second predictive model configured to receive the synthetic path descriptor and predict a second target molecule descriptor corresponding to the target material.

8. The method of claim 7, wherein the predicting of the one or more synthetic path descriptor candidates comprises:
applying the first target molecule descriptor to the first predictive model;
acquiring the synthetic path descriptor predicted by the first predictive model based on the first target molecule descriptor;
determining whether a candidate material corresponding to the synthetic path descriptor is a starting material of which a chemical characteristic and a structure are known;
predicting the second target molecule descriptor by applying the synthetic path descriptor to the second predictive model in response to a determination that the candidate material is the starting material; and
predicting the one or more synthetic path descriptor candidates based on whether the first target molecule descriptor matches the second target molecule descriptor.

9. The method of claim 8, wherein the applying of the first target molecule descriptor to the first predictive model comprises:
classifying one or more characters representing an atom type of the target material and one or more characters representing a chemical bond of the target material in the first target molecule descriptor as tokens; and
applying at least a portion of the tokens to the first predictive model.

10. The method of claim 8, wherein the determining of whether the candidate material is the starting material comprises:
determining whether the candidate material is the starting material using data registered in a material database.

11. The method of claim 8, further comprising:
removing, in response to a determination that the candidate material is not the starting material, the synthetic path descriptor corresponding to the candidate material from the synthetic path descriptor candidates.

12. The method of claim 1, further comprising:
providing a synthesis recipe corresponding to the target material based on the synthetic path descriptor candidates.

13. The method of claim 1, further comprising:
receiving the multi-step synthetic path data corresponding to chemical materials; and
converting the multi-step synthetic path data into the synthetic path descriptor in a form of a character string.

14. A non-transitory computer-readable storage medium storing instructions that, when executed by at least one processor, cause the at least one processor to perform a method of predicting a synthetic path, the method comprising:
receiving a target molecule descriptor corresponding to a target material;
predicting one or more synthetic path descriptor candidates representing multi-step synthetic paths corresponding to the target molecule descriptor using a neural network-based predictive model, the neural network-based predictive model being trained based on a synthetic path descriptor obtained by converting multi-step synthetic path data; and
outputting the one or more synthetic path descriptor candidates.

15. An apparatus for predicting a synthesis path, the apparatus comprising:
a user interface configured to receive a target molecule descriptor corresponding to a target material;
at least one memory storing at least one program; and
at least one processor configured to execute the at least one program to:
predict one or more synthetic path descriptor candidates representing multi-step synthetic paths corresponding to the target molecule descriptor using a neural network-based predictive model, the neural network-based predictive model being trained based on a synthetic path descriptor obtained by converting multi-step synthetic path data; and
provide a synthesis recipe corresponding to the target material based on the one or more synthetic path descriptor candidates.

16. The apparatus of claim 15, wherein the synthetic path descriptor comprises one or more tokens among:
a molecular structure descriptor representing molecular structure information of reactants;
a notation character for distinguishing synthesis steps before and after synthesis of the multi-step synthetic paths;
a delimiting parenthesis for defining a synthesis order of the reactants used for each synthesis step of the multi-step synthetic paths;
a separator for distinguishing between the molecular structure descriptor and the delimiting parenthesis or distinguishing between a plurality of molecular structure descriptors; and
a reaction descriptor corresponding to a reaction scheme used for each synthesis step of the multi-step synthetic paths.

17. The apparatus of claim 15, wherein the neural network-based predictive model comprises an encoder and a decoder that are trained using the synthetic path descriptor in which a portion of tokens are masked.

18. The apparatus of claim 15, wherein the neural network-based predictive model comprises:
a first encoder configured to extract embedding information from the target molecule descriptor;
a second encoder that is trained to extract a first feature from the embedding information and a sequence comprising first tokens of a portion among tokens constituting the synthetic path descriptor; and
a decoder that is trained to restore a character string corresponding to the synthetic path descriptor based on the first feature and information on second tokens other than the first tokens among the tokens constituting the synthetic path descriptor, and
wherein the neural network-based predictive model is trained by updating one or more weights of the neural network-based predictive model based on a difference between the synthetic path descriptor and the character string.

19. The apparatus of claim 17, wherein the neural network-based predictive model comprises:
a first predictive model that receives a first target molecule descriptor corresponding to the target material and predicts the synthetic path descriptor corresponding to the target material; and
a second predictive model that receives the synthetic path descriptor and predicts a second target molecule descriptor corresponding to the target material.

20. The apparatus of claim 19, wherein the at least one processor is configured to:
apply the first target molecule descriptor to the first predictive model;
acquire the synthetic path descriptor predicted by the first predictive model based on the first target molecule descriptor;
determine whether a candidate material corresponding to the synthetic path descriptor is a starting material of which a chemical characteristic and a structure are known;
predict the second target molecule descriptor by applying the synthetic path descriptor to the second predictive model in response to a determination that the candidate material is the starting material; and
predict the one or more synthetic path descriptor candidates based on whether the first target molecule descriptor matches the second target molecule descriptor.
